(19) United States
(12) Patent Application Publication
(10) Pub. No.: US 2024/0050958 A1
(43) Pub. Date: Feb. 15, 2024

(54) MECHANICAL HEATING CONTROL

(71) Applicant: HP Health Solutions Inc., Palo Alto, CA (US)

(72) Inventors: Duane A. Koehler, Vancouver, WA (US); Devin Knowles Michael, Vancouver, WA (US); Devin Nichlos Koepl, Vancouver, WA (US)

(73) Assignee: HP Health Solutions Inc., Palo Alto, CA (US)

(21) Appl. No.: 18/268,083

(22) PCT Filed: Dec. 22, 2020
(86) PCT No.: PCT/US2020/066627

§ 371 Date: Jun. 16, 2023

(51) Int. Cl. B01L 7/00; G01N 33/48

(52) U.S. Cl. CPC B01L 7/52; G01N 33/48; B01L 2200/147; B01L 2300/1827; G01N 2035/00346

(57) ABSTRACT

An example device comprises: a heater; a temperature sensor to monitor changes in temperature caused by the heater; a mechanical device to move the heater between a sample position and a rest position; and a circuit communicatively coupled to the heater, the temperature sensor, and the mechanical device. The circuit is to: control the mechanical device to move the heater to the sample position; control the heater to heat at the sample position; monitor, via the temperature sensor, the temperature; and in response to the temperature meeting a temperature condition, control the mechanical device to move the heater from the sample position to the rest position.

### BACKGROUND

Sample heating in devices that effect biological or chemical processes in sample preparation cartridge modules can be crucial, for example in sample preparation devices.

### BRIEF DESCRIPTION OF THE DRAWINGS

Reference will now be made, by way of example only, to the accompanying drawings in which:

FIG. 1 is a block diagram of an example device with mechanical heating control.

FIG. 2A is a perspective view of an example sample preparation device that incorporates aspects of the device of FIG. 1.

FIG. 2B is a block diagram of the device of FIG. 2A.

FIG. 3 is a block diagram of the device of FIG. 2A showing cassette and sample preparation cartridge module in a heating position.

FIG. 4A and FIG. 4B show heaters of the device of FIG. 2A in rest positions and sample positions respectively, as well as a sample preparation cartridge module at the sample positions.

FIG. 5 is a block diagram showing control components of the device of FIG. 2A, such as a processor, to implement mechanical heating control.

FIG. 6 is a flow diagram of an example method for mechanical heating control.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, or the like, that can be present in a biological sample. A “biological sample” can refer to a fluid or a dried or lyophilized material obtained for analysis from a living or deceased organism. Isolating the biological component from other components of the biological sample can permit subsequent analysis without interference and can increase an accuracy of the subsequent analysis. In addition, isolating a biological component from other components in a biological sample can permit analysis of the biological component that would not be possible if the biological component remained in the biological sample. In this context, “Isolation” can also be referred to as “purification”, whereby biological component may be separated from the rest of the biological sample after introduction to a sample preparation cartridge module interchangeably referred to hereafter as a sample container, a sample dispensing container, a cartridge module, and the like. It will be understood that the isolated biological component may be output in association with (e.g., bound to) particulate substrate and a reagent solution, or the like. The isolation or purification refers to the separation of the biological component from other components of the biological sample with which it was originally introduced in the cartridge module, but it does not mean that the biological component is completely isolated when it is dispensed. For example, isolation refers to the fact that the biological component is sufficiently separated or “purified” from other components of the original biological sample to facilitate further processing such as detection and/or amplification.

Many isolation techniques can include repeatedly dispersing and re-aggregating samples. The repeated dispersing and re-aggregating can result in a loss of a quantity of the biological component. Furthermore, isolating a biological component with some of these techniques can be complex, time consuming, and labor intensive and can result in less than maximum yields of the isolated biological component. Such Isolation techniques are done using specific devices.

Obtaining precise biological sample preparation devices can be challenging due to many moving parts present in the devices, for example to move a carriage holding a cartridge of sample dispensing containers relative to sample receiving wells. The cartridge may hold a plurality of the sample dispensing containers or sample preparation devices or sample preparation cartridge modules which contain different respective biological samples.

During the isolation process, The sample dispensing containers or sample preparation devices or sample preparation cartridge modules may heat the samples to perform for example, lysis on cells in the biological samples to release biological component of interest, coming from the biological sample, may be a nucleic acid (such as DNA or RNA). Resulting sample fluid may be drawn through a fluid density gradient in the sample dispensing containers and dispensed into sample receiving wells, which may be transferred to further analytical assay such as, for example, a Polymerase Chain Reaction (PCR).

However, as initial quantities of the biological component of interest present in the biological sample, may be small, precise dispensing of the component of interest from the sample dispensing containers into the sample receiving wells should occur so as to not lose any content and/or to prevent cross-contamination between samples. As such a precise determination of a position of a shuttle and/or well carriage, that holds the sample receiving wells, relative to the carriage is important, and vice versa.

In some examples, the device of the present disclosure is a device that can be used to prepare sample to be used in a process of preparing samples for a PCR (polymerase chain reaction) assay. PCR assays are processes that can rapidly copy millions to billions of copies of a very small DNA or RNA sample. PCR can be used for many different application, included sequencing genes, diagnosing viruses, identifying cancers, and others. In the PCR process, a small sample of DNA or RNA is combined with reactants that can form copies of the DNA or RNA.

As described herein, the biological sample comprises a biological component. In some examples, the biological component of interest, coming from the biological sample, may be a nucleic acid (such as DNA or RNA). A particulate substrate can be configured to be associated with the biological component, to isolate the biological component from the biological sample. In one example, the particulate substrate comprises paramagnetic beads and/or any magnetizing particle and/or magnetizing microparticles. In one example, the biological component comprises nucleic acids such as DNA and/or RNA that may be extracted from the biological sample by lysing, bound to magnetic particulate substrate, and separated from the lysate and dragged towards an output by an externally generated (para)magnetic force. Lysate may refer to the fluid containing the material resulting from the lysis of a biological sample. Such lysis may release the biological component that is contained therein. Lysing itself may include mixing and/or heating the biological sample, chemically lysing the biological sample, and/or a combination of the foregoing.

Hence, as will now be apparent, sample heating in such devices, that process biological samples using sample preparation cartridge modules, and the like, can be crucial.

As such, provided herein is a device that that includes a heater attached to a mechanical device that moves the heater between a sample position, to heat a sample preparation cartridge module at the sample position, and a rest position and/or a cooling position, at which the heater does not heat the sample preparation cartridge module. The heater, and/or a chassis thereof, may be generally shaped to engage the sample preparation cartridge module. The device includes a temperature sensor that monitors changes in temperature caused by the heater. A circuit generally controls the mechanical device to move between the sample position and the rest position by monitoring temperatures measured by the temperature sensor. The device may be incorporated into a larger sample preparation device sample preparation cartridge module that processes biological samples, as described above to, perform lysis.

When the heater is in the sample position (e.g. to heat a sample preparation cartridge module to perform lysis on cells in the sample preparation cartridge module), the circuit, in response to the temperature meeting a temperature condition, controls the mechanical device to move the heater from the sample position to the rest position, for example to stop heating the sample. The temperature condition may be that the temperature monitored by the temperature sensor has reached or exceeded a threshold temperature above which it is undesirable to heat a sample, as at some temperature, above the threshold temperature, biological components of interest in the sample may be damaged and/or the device may be damaged and/or other components of a larger sample preparation device into which the device is incorporated may be damaged. Such a rise in temperature may be due to a fault in the heater, and the like. The circuit may be further to control the heater to stop heating (e.g. turn off) based on the temperature meeting the temperature condition.

In some examples, the circuit may be further to control a position of the heater, via the mechanical device, to control a heating cycle of the sample, for example moving the heater closer to, and further from, the sample preparation cartridge module and/or controlling mechanical pressure or mechanical force of the heater on the sample preparation cartridge module (e.g. at the sample position). Such a heating cycle may be further controlled by the circuit turning the heater on, off and/or to different power levels.

In some examples, the device may comprise a pair of heaters, attached to respective mechanical devices, that include respective temperature sensors. The pair of heaters may be shaped to engage a sample preparation cartridge module, at a respective sample positions, from opposite directions. One, or both, of the heaters may be controlled to engage the sample preparation cartridge module to control a heating cycle.

A first aspect of the present specification provides a device comprising: a heater; a temperature sensor to monitor changes in temperature caused by the heater; a mechanical device to move the heater between a sample position and a rest position; and a circuit communicatively coupled to the heater, the temperature sensor, and the mechanical device, the circuit to: control the mechanical device to move the heater to the sample position; control the heater to heat at the sample position; monitor, via the temperature sensor, the temperature; and in response to the temperature meeting a temperature condition, control the mechanical device to move the heater from the sample position to the rest position.

At the device of the first aspect, the circuit may be further to, in response to the temperature meeting the temperature condition, control the heater to stop heating.

At the device of the first aspect, the temperature sensor may be located at the heater.

At the device of the first aspect, the mechanical device may comprise a robotic arm or an arm and a servomotor, and the heater may be located at an end effector position of the mechanical device.

At the device of the first aspect, the heater may be further to heat a sample preparation cartridge module at the sample position to effect a biological or chemical process in the sample preparation cartridge module.

A second aspect of the present specification provides a device comprising: a heater; a temperature sensor to monitor changes in temperature caused by the heater; a mechanical device to move the heater between a sample position and a rest position; and a circuit communicatively coupled to the heater, the temperature sensor, and the mechanical device, the circuit to: control the heater to heat; monitor, via the temperature sensor, the temperature; and control the mechanical device to adjust a position of the heater, relative to the sample position and the rest position, based on the temperature, to control a heating cycle.

At the device of the second aspect, the circuit may be further to control the mechanical device to adjust the position of the heater, to control the heating cycle, by adjusting a distance of the heater from the sample position.

At the device of the second aspect, the circuit may be further to control the mechanical device to adjust the position of the heater, to control the heating cycle, by adjusting mechanical pressure or mechanical force at the sample position.

At the device of the second aspect, the circuit may be further to control the mechanical device to adjust the position of the heater, to control the heating cycle, by controlling a time period that the heater is at the sample position.

At the device of the second aspect, the circuit may be further to control the heating cycle by controlling power to the heater.

A third aspect of the present specification provides a method comprising: monitoring, at a device, via a temperature sensor, changes in temperature caused by a heater attached to a mechanical device, the mechanical device to move the heater between a sample position and a rest position; controlling, at the device, power to the heater, based on the temperature relative to a target temperature and a threshold temperature; and controlling, at the device, the mechanical device to move the heater, relative to the sample position, based on the temperature relative to the target temperature and the threshold temperature.

At the method of the third aspect, controlling the mechanical device to move the heater, based on the temperature, relative to the target temperature and the threshold temperature, may comprise: in response to the temperature reaching the threshold temperature, moving the heater away from the sample position until the temperature is at or below the target temperature.

At the method of the third aspect, controlling the power to the heater, based on the temperature relative to the target temperature and the threshold temperature, may comprise: in response to the temperature reaching the threshold temperature, turning the heater off until the temperature is at or below the target temperature.

The method of the third aspect may further comprise, in response to the temperature reaching or exceeding the threshold temperature for a given time period: moving the heater away from the sample position; and turning the heater off.

The method of the third aspect may further comprise, in response to the temperature reaching or exceeding the threshold temperature for a given time period: controlling a notification device to provide a notification of a fault.

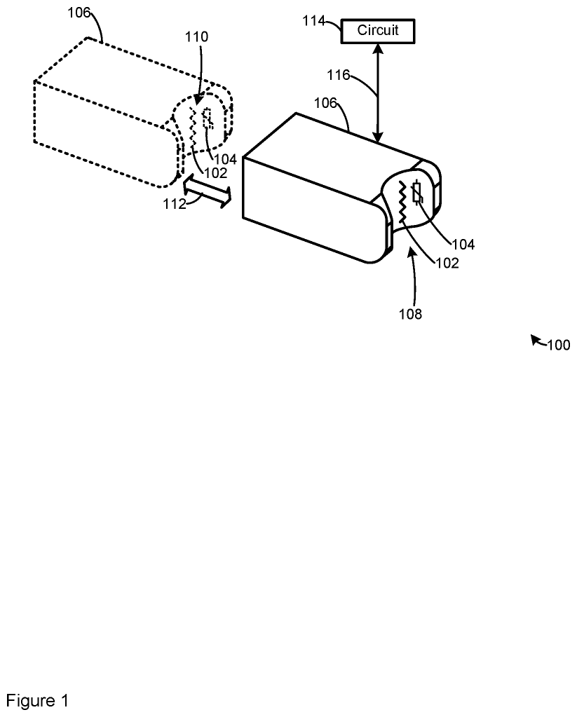

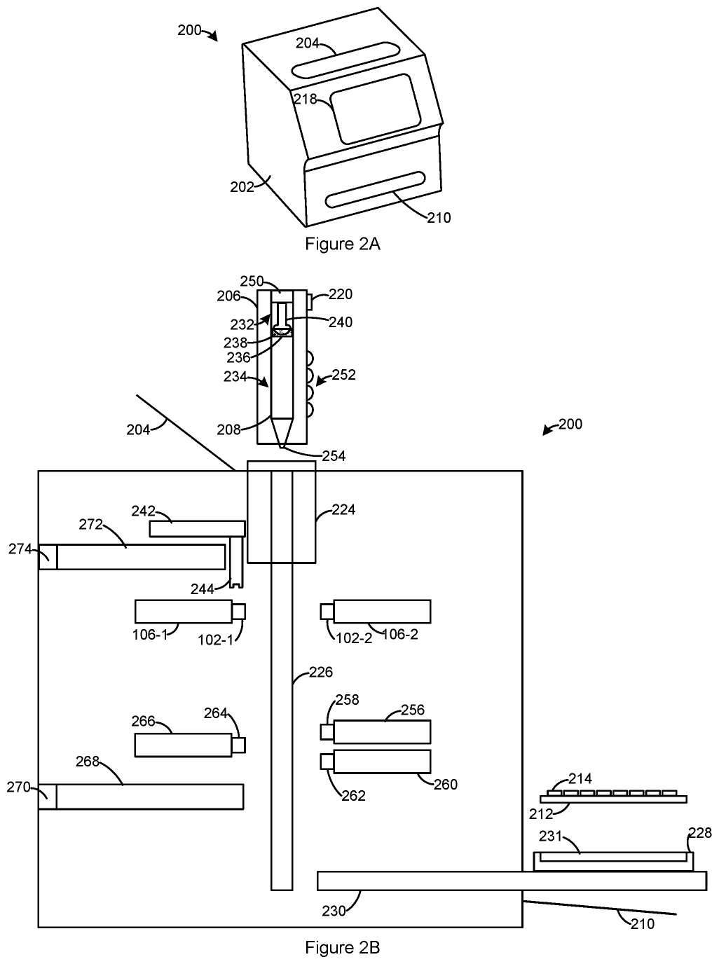

FIG. 1 is a block diagram of an example device 100 with mechanical heating control. While describe in more detail below, it is understood that components of the device 100 may be components of a larger device used for medical testing of samples including, but not limited to, sample preparation. In particular, the device 100 may be used in a larger sample preparation device to process s of samples in sample preparation cartridge modules. Such a sample preparation device is described below with respect to FIG. 2A and FIG. 2B.

The device 100 generally comprises a heater 102, represented in FIG. 1 as a resistor, and a temperature sensor 104, represented in FIG. 1 as a thermistor. The device 100 further comprises a mechanical device 106 to move the heater between a sample position 108 and a rest position 110. As depicted, the mechanical device 106 and the heater 102 (as well as the temperature sensor 104) are in the sample position 108, with the mechanical device 106 and the heater 102 (as well as the temperature sensor 104) in the rest position 110 shown in outline and/or dashed lines, with movement between the positions 108, 110 represented by an arrow 112.

As depicted, the heater 102 and/or the mechanical device 106 is shaped to engage a sample preparation cartridge module (not depicted) at the sample position 108. As previously mentioned, such a sample preparation cartridge module may alternatively be referred to as a cartridge module, a sample container, a sample dispensing container, and the like.

For example, the heater 102 and/or the mechanical device 106 may include a chassis, and the like, that is shaped complementary to a sample preparation cartridge module located at the sample position 108. In a particular example, as depicted, a chassis of the heater 102 and/or the mechanical device 106 is concave to engage a cylindrically shaped sample preparation cartridge module at the sample position 108. However, the heater 102 and/or the mechanical device 106 may be any suitable shape to heat a sample preparation cartridge module (e.g. which may also be of any suitable shape).

In general, the device 100 further comprises a circuit 114 that is communicatively coupled to the heater 102, the temperature sensor 104, and the mechanical device 106. While electrical connections from the circuit 114 to the various other components are not explicitly depicted, such electrical connections and/or communicative couplings are represented by a double ended arrow 116 between the circuit 114 and the mechanical device 106 which may represent wiring, and the like, of the circuit 114 to the various other components.

The circuit 114 is generally to control the mechanical device 106 to move the heater 102 to the sample position 108 (e.g. from the rest position 110), for example to engage a sample preparation cartridge module at the sample position 108; control the heater 102 to heat at the sample position 108, for example to heat a sample preparation cartridge module at the sample position 108; monitor, via the temperature sensor 104, temperature which may represent a temperature of the sample preparation cartridge module and/or the heater 102; and in response to the temperature meeting a temperature condition, control the mechanical device 106 to move the heater 102 from the sample position 108 to the rest position 110.

The temperature conditions may alternatively be referred to as interlock conditions as, in response to the temperature meeting a temperature condition, such as a threshold temperature and/or an overshoot temperature, the controlling of the mechanical device 106 to move the heater 102 from the sample position 108 to the rest position 110 may act as an interlock to prevent damage to a sample preparation cartridge module at the sample position 108 and/or sample contained in the sample preparation cartridge module.

While not depicted, it is understood that the device 100 further comprises a power source, which powers the components of the device 100, including, but not limited to, the heater 102, such that the heater 102 is powered to perform heating. Such a power source may include, but is not limited to, a connection to a mains power supply and/or batteries, and the like,

The heater 102 may comprise any suitable heating device including, but not limited to, the depicted resistor incorporated into a chassis, and the like, which may be shaped to engage a sample preparation cartridge module. It is generally understood that the heater 102 is positioned at the device 100 to be adjacent and/or against (e.g. via a wall of a chassis of the heater 102 and/or the mechanical device 106) a sample preparation cartridge module at the sample position 108. It is understood that the chassis of the heater 102 and/or the mechanical device 106 may comprise thermally conducting material, such as any suitable combination of metal and/or a thermally conducting plastic, and the like, to conduct heat from the heater 102 to a sample preparation cartridge module.

In some examples, a portion of the material of the chassis of the heater 102 and/or the mechanical device 106 that resides against a sample preparation cartridge module may be (e.g. optionally) at least partially elastic, and the like, to at least partially mold around a sample preparation cartridge module at the sample position 108, to assist with thermal conduction from the heater 102 to the sample preparation cartridge module.

In general, the heater 102 may be controlled to on on-state or an off-state (e.g. on or off) by the circuit 114. However, the heater 102 may alternatively be controlled to different heating levels in the on-state, to heat a sample preparation cartridge module at the sample position 108 to different temperatures, as monitored by the temperature sensor 104. Alternatively, the heater 102 may be controlled between an on-state and off-state to heat a sample preparation cartridge module at the sample position 108 to given temperature and/or different temperatures using pulse width modulation (PWM) control of the heater 102 (e.g. to control, by the circuit 114, relative time periods that the heater 102 is on or off).

However, the heater 102 may also heat a sample preparation cartridge module at the sample position 108 to different temperatures by the circuit 114 controlling the mechanical device 106 to move the heater 102 to different positions, relative to the sample position 108. For example, the closer the heater 102 is to the sample position 108, the more heating of a sample preparation cartridge module at the sample position 108 may occur. Alternatively, such control the mechanical device 106 may also occur according to PWM control (e.g. to control, by the circuit 114, relative time periods that the heater 102 is at the sample position 108 or the rest position 110).

Regardless, it is understood that the heater 102 is movable relative to the sample position 108.

It is further understood that the heater 102 may be controlled to a given temperature (e.g. by the circuit 114) in a feedback loop, based on feedback from the sensor 104.

The temperatures sensor 104 (hereafter interchangeably referred to as the sensor 104) may comprise any suitable temperature sensing device and/or circuit including, but not limited to, the depicted thermistor incorporated into a chassis, and the like, at which the heater 102 is also incorporated. It is generally understood that the sensor 104 is positioned at the device 100 to be adjacent and/or against (e.g. via a wall of a chassis of the heater 102 and/or the mechanical device 106) a sample preparation cartridge module at the sample position 108. As such, the sensor 104 generally measures temperature changes due to the heater 102 being turned on or off by the circuit 114 (and/or otherwise being generally controlled by the circuit 114).

As depicted the temperature sensor 104 is depicted as being at a chassis, and the like, at which the heater 102 is also incorporated, and hence, as depicted, the temperature sensor 104 also moves with the heater 102. Put another way, the temperature sensor 104 may be located at the heater 102.

However, in other examples the temperature sensor 104 may be at a fixed position, for example at the sample position 108, and include components for engaging a sample preparation cartridge module at the sample position 108. For example, the temperature sensor 104 may be incorporated into a spring loaded arm which causes the sensor 104 to reside against a sample preparation cartridge module at the sample position 108.

In yet other examples, the temperature sensor 104 may be remote from the mechanical device 106 and/or the heater 102 and/or sample position 108 and may comprise, for example, a remote temperature sensor, including, but not limited to, an infrared (IR) temperature sensor, that reads the temperature of the sample preparation cartridge module by sensing IR signature.

The mechanical device 106 may comprise any suitable mechanical device which moves the heater 102 between the sample position 108 and the rest position 110, including, but not limited to, a robotic arm, an arm attached to a servomotor, and the like. An optical encoder, and the like, at the robotic arm and/or the servomotor may be used to control the mechanical device 106 between the sample position 108 and the rest position 110.

Regardless, the heater 102 may be understood to be located at an end effector position of the mechanical device 106 (e.g. an end of the mechanical device 106 that interacts with an environment around the mechanical device 106 including, but not limited to, a sample preparation cartridge module at the sample position 108).

While the mechanical device 106 is depicted as controlling the heater 102 to the sample position 108 and the rest position 110, it is understood that the mechanical device 106 may alternatively to be controlled (e.g. by the circuit 114) to move the heater 102 to any suitable position between the sample position 108 and the rest position 110, for example to control a position of the heater 102 relative to a sample preparation cartridge module at the sample position 108 to control a heating cycle at the sample preparation cartridge module.

Such a heating cycle may include, but is not limited to, respective times that the sample preparation cartridge module is at respective given temperatures as measured by the sensor 104, ramp times to reach such temperatures, cooling times, and the like.

Furthermore, in some examples, the mechanical device 106 may be controlled (e.g. by the circuit 114) to adjust the position of the heater 102 by adjusting mechanical pressure or mechanical force at the sample position 108. For example, a robotic arm and/or a servomotor, and the like, of the mechanical device 106 may be controlled (e.g. by the circuit 114) to position the heater 102 against a sample preparation cartridge module at the sample position 108 and apply mechanical pressure and/or mechanical force to the sample preparation cartridge module at the sample position 108, for example to change and/or control thermal contact and/or thermal conduction between the heater 102 and a sample preparation cartridge module. In some of these examples, the mechanical device 106 may be spring loaded to apply mechanical pressure or mechanical force at the sample position 108.

Put another way, the circuit 114 may further to control the mechanical device 106 to adjust the position of the heater 102, to control a heating cycle, by adjusting mechanical pressure or mechanical force at the sample position 108.

For example, while not depicted, the device 100 and/or the mechanical device 106 may include a pressure sensor and/or a force sensor to measure pressure and/or force applied by the mechanical device 106, and such a pressure sensor and/or a force sensor may be used to control the pressure and/or force applied by the mechanical device 106 in a feedback loop with the circuit 114. Alternatively, the pressure and/or force applied by the mechanical device 106 may be controlled via optical encoder positions of a servomotor of the mechanical device 106 and/or in any other suitable manner.

A heating cycle may also be controlled by controlling a time period that the heater 102 is at the sample position 108. For example, the circuit 114 may be further to control the mechanical device 106 to adjust the position of the heater 102, to control a heating cycle, by controlling a time period that the heater 102 is at the sample position 108. In particular, the circuit 114 may be further to control a heating cycle by controlling power to the heater 102, such that the heater 102 is turned on for a given time period, to a power level or different power levels, and then turned off.

The circuit 114 may comprise any suitable combination of components to control the heater 102 and the mechanical device 106 based on temperature measured by the sensor 104. For example, the circuit 114 may comprise connections to the heater 102, a power source to power the heater 102, switches and/or other components to turn the heater 102 on or off and/or to adjust a current and/or power to the heater 102 to control the heater 102 to different heating levels. In general, the circuit 114 is to control the heater 102 to a given temperature and/or given temperatures, for example to control a heating cycle of a sample preparation cartridge module at the sample position 108, as well as provide interlock functionality as described above, though the interlock functionality may be optional in some examples.

In some examples, the circuit 114 may comprise a proportional-integral-derivative controller (PID) controller to control the heater 102 to a given temperature and/or given temperatures, for example to control a heating cycle and/or heating cycles of a sample preparation cartridge module at the sample position 108 based on feedback from the sensor 104.

The circuit 114 may further comprise connections to the mechanical device 106, and/or a servomotor thereof, to control the mechanical device 106 to move the heater 102 relative to the sample position 108. In a simple example, the circuit 114 may control the mechanical device 106 to move the heater 102 to the sample position 108 or the rest position 110. For example, the circuit 114 may control the mechanical device 106 to move the heater 102 to the sample position 108 to heat a sample preparation cartridge module, and the circuit 114 may control the mechanical device 106 to move the heater 102 to the rest position 110 to cool the sample preparation cartridge module.

In some of these examples, the circuit 114 may control the heater 102 to be on when the mechanical device 106 is controlled to move the heater 102 to the sample position 108 for example to heat the sample preparation cartridge module to a given temperature and/or for a given period of time to effect lysis and the like at the sample preparation cartridge module. The circuit 114 may then control the heater 102 to be off when the mechanical device 106 is controlled to move the heater 102 to the rest position 110 for example after the given period of time. However, the circuit 114 may control the heater 102 to be off when the mechanical device 106 is controlled to move the heater 102 to the rest position 110 based on the temperature, measured by the sensor 104, meeting a temperature condition.

The temperature condition may be that the temperature monitored by the temperature sensor 104 has reached or exceeded a threshold temperature. In some examples such a threshold temperature may comprise a temperature above which it is undesirable to heat a sample, as at some temperature, above the threshold temperature, biological components of interest in the sample may be damaged and/or the device 100 may be damaged and/or other components of a larger sample preparation device into which the device 100 is incorporated may be damaged. Such a rise in temperature to the threshold temperature may be due to a fault in the heater 102 and/or the circuit 114 and/or the sample preparation cartridge module any other suitable condition. In some examples, such a threshold temperature may be referred to as an overheat temperature.

Put another way, the heater 102 may be to heat a sample preparation cartridge module at the sample position 108 to effect a biological or chemical process in the sample preparation cartridge module, and the temperature condition may be associated with preventing damage in the biological or chemical process. In such examples, the biological or chemical process may be lysis to rupture cells in a sample held by the sample preparation cartridge module, and the temperature condition may be associated with preventing damage to biological components of interest in the cells. As such, the temperature condition may be the temperature measured by the sensor 104 reaching or exceeding a threshold temperature such that, in response to the temperature measured by the sensor 104 reaching or exceeding a threshold temperature, the circuit 114 controls the mechanical device 106 to move the heater 102 from the sample position 108 to the rest position 110 (e.g. and turn the heater 102 off). In these examples the threshold temperature may be less than a temperature at which damage in the biological or chemical process occurs, for example to prevent a sample in the container from reaching such a temperature.

Hence, in general, the circuit 114 may be to control the heater 102 to heat the sample preparation cartridge module to a given target temperature to effect the biological or chemical process, and also to control the heater 102 prevent the heater 102 from damaging a biological sample on which the biological or chemical process is being performed.

However, the temperature condition may alternatively be associated with preventing device damage. For example, the temperature condition may be that the temperature monitored by the temperature sensor 104 has reached or exceeded a threshold temperature above which the device 100 may be damaged and/or other components of a larger sample preparation device into which the device 100 is incorporated may be damaged. Such a rise in temperature to the threshold temperature may be due to a fault in the heater 102 and/or the circuit 114 and/or the sample preparation cartridge module any other suitable condition.

In some examples, the temperature condition may be time based; for example, the temperature condition may be that the temperature monitored by the temperature sensor 104 has reached or exceeded a threshold temperature for a given time period, for example 5 seconds, 10 seconds, and the like, and/or any other suitable time period.

Furthermore, as described above, the circuit 114 may be further to, in response to the temperature meeting the temperature condition, control the heater 102 to stop heating and/or turn off, for example, to stop heating a sample preparation cartridge module, which may occur in addition to moving the heater 102 to the rest position 110.

However, the circuit 114 may alternatively adjust a position of the mechanical device 106 to move the heater 102 to different positions between the sample position 108 and the rest position 110 to assist with controlling a heating cycle of a sample preparation cartridge module at the sample position 108 based on feedback from the sensor 104. For example, the circuit 114 may adjust a position of the mechanical device 106 to move the heater 102 towards or away from a sample preparation cartridge module at the sample position 108 and concurrently increase or decrease power to the heater 102 to control an amount of heat being emitted by the heater 102, for example to control a heating cycle.

Put another way, the circuit 114 may be to: control the heater 102 to heat; monitor, via the temperature sensor 104, temperature; and control the mechanical device 106 to adjust a position of the heater 102, relative to the sample position 108 and the rest position 110, based on the temperature, to control a heating cycle. For example, the circuit 114 may be further to control the mechanical device 106 to adjust the position of the heater 102, to control the heating cycle, by adjusting a distance of the heater 102 from the sample position 108.

The circuit 114 may include any suitable combination of switches, electrical circuits, and the like which implement functionality as described herein. In some examples, the circuit 114 may further include a general-purpose processor and/or controller or special purpose logic, such as a microprocessor and/or microcontroller (e.g. a central processing unit (CPU) and/or a graphics processing unit (GPU) an integrated circuit or other circuitry), an application specific integrated circuit (ASIC), a Field Programmable Gate Array (FPGA), a programmable array logic (PAL), a programmable logic array (PLA), a programmable logic device (PLD), and the like. Hence, functionality of the circuit 114 may be implemented as a combination of hardware (e.g. a CPU, a GPU, etc.) and software (e.g., programming such as machine- or processor-executable instructions, commands, or code such as firmware, a device driver, programming, object code, etc. as stored on hardware). Alternatively, the circuit 114 may be implemented as a hardware element with no software elements (e.g. such as an ASIC, an FPGA, a PAL, a PLA, a PLD etc.).

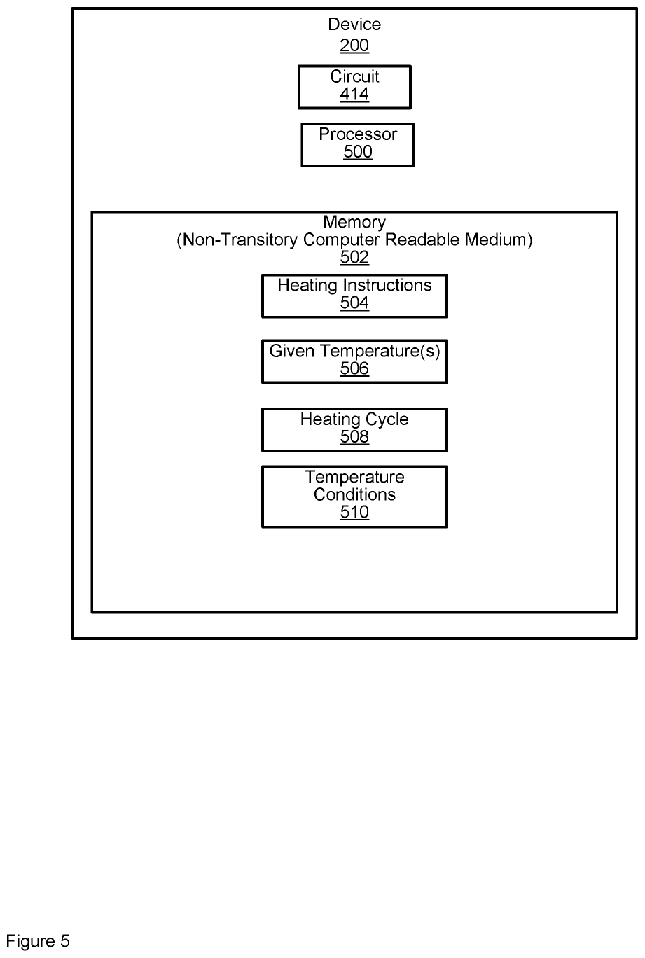

While not depicted, given temperatures, temperature conditions, threshold temperatures, heating cycles, positions of the mechanical device 106, and the like, may be stored at a memory of the device 100 (e.g. see FIG. 5), along with instructions for controlling the circuit 114 and/or a processor thereof (e.g. when the circuit is implemented as combination of hardware and software). Such a memory may include, but is not limited to, any suitable combination of a volatile computer-readable medium (e.g., volatile RAM, a processor cache, a processor register, etc.), a non-volatile computer-readable medium (e.g., a magnetic storage device, an optical storage device (e.g. a Digital Versatile Disc (DVD), a paper storage device, flash memory, read-only memory, non-volatile RAM, etc.), and/or the like.

Attention is next directed to FIG. 2A and FIG. 2B which respectively depict a perspective view and a block diagram of an example sample preparation device 200 that incorporates aspects of the device 100 of FIG. 1. As depicted, the sample preparation device 200 (interchangeably referred to hereafter as the device 200) includes a chassis 202 that includes a cassette access door 204 for loading a cassette 206 that includes a sample preparation cartridge module 208 and/or sample preparation cartridge modules 208 therein, the sample preparation cartridge module 208 holding a sample for testing as described hereafter. While the sample preparation cartridge modules 208 are depicted herein as being in an elongate shape and/or in the form of a column, the sample preparation cartridge modules 208 may be any suitable shape.

The chassis 202 further includes a well access door 210 for loading a well holder 212 containing a well 214 and/or wells for receiving processed samples dispensed from the sample preparation cartridge module 208 after processing by the device 200. While only one sample preparation cartridge module 208 is depicted, and eight wells 214, it is understood that the cassette 206 may hold a same number of sample preparation cartridge modules 108 as there are wells 214 at the well holder 212. For example, as depicted, there may be eight sample preparation cartridge modules 208 and eight wells 214. Furthermore, while the cassette 206 is depicted in an end view showing only one sample preparation cartridge module 208, and the well holder 212 is shown in a front view showing eight wells 214, the components of the device 200 may cause the cassette 206 and the well holder 212 to be loaded into the device 200 in any suitable relative orientation including, but not limited to, about parallel to one another such that a line of the sample preparation cartridge module 208 is about aligned with a line of the wells 214.

As depicted, the device 200 further comprises an input device 218, such as a touch screen display, and the like, which may be used to control the device 200 into a loading mode, which causes the cassette access door 204 and the well access door 210 to open such that the cassette 206 and the well holder 212, with the wells 214, may be manually loaded into the device 200. Hence, it is understood that a sample preparation cartridge module 208 is loaded with a sample 238 (e.g. such as a biological sample retrieved from a human by medical personnel), and the like, via a port 220. The input device 218 may also be used to set given temperatures to which the sample preparation cartridge module 208 is to be heated and/or a heating cycle of the sample preparation cartridge module 208 and/or a heating/mixing cycle (e.g. setting mixing speeds of a mixer of the device 200).

In the loading mode, a cassette carriage 224 of the device 200 is raised along a vertical carriage guide 226 to at least partially emerge from an opening that is normally covered by the cassette access door 204. The cassette 206 is then manually loaded into the cassette carriage 224.

Similarly, in the loading mode, a well carriage (e.g. a shuttle) 228, which moves linearly on a horizontal carriage guide (e.g. a planar surface) 230, is moved out of an opening that is normally covered by the well access door 210, for example by moving and/or rotating and end of horizontal carriage guide 230 at which the well carriage 228 is located in the loading mode, out of the opening. The well holder 212 is then manually loaded into a complementary shaped depression and/or holder 231 in the well carriage 228. While the terms vertical and horizontal are used herein with regards to a position of the device 200 in a normal use mode, such terms are meant for ease of description only and/or to indicate relative positions of components of the device 200 (e.g. the guides 226, 230 may be about perpendicular to each other as one is vertical and the other horizontal, but may be in any suitable orientation).

Once loaded, the cassette carriage 224 moves the cassette 206 into different positions in the device 200 (e.g. closing the door 204), for example along the vertical carriage guide 226, to process the sample 238 before dispensing the sample 238 from the sample preparation cartridge module 208 into a well 214. As such, while not depicted, the device 200 is further understood to include a motor and/or a servomotor, and the like, to move the cassette carriage 224 into different positions along the vertical carriage guide 226.

Similarly, once loaded, the horizontal carriage guide 230 moves inside the device 200 (e.g. closing the door 210) and the well carriage 228 is moved into a position to receive the sample 238 from the sample preparation cartridge module 208 into a corresponding well 214. When there a plurality of sample preparation cartridge modules 208 holding respective samples 238, once the samples 238 are processed, the well carriage 228 is moved into respective positions to receive respective samples 238 dispensed from respective sample preparation cartridge modules 208 into corresponding wells 214. As such, the well carriage 228 may be positioned at an angle relative to the cassette carriage 224 and/or the cassette 206 such that different sample preparation cartridge modules 208 align with different wells 214 at different positions of the well carriage 228.

As such, while not depicted, the device 200 is further understood to include motors and/or a servomotors, and the like, to move the horizontal carriage guide 230 into and out of the device 200, and to linearly move the well carriage 228 along the horizontal carriage guide 230.

While not depicted, the device 200 may further include respective components for opening and closing the doors 204, 210.

To effect processes of the device 200, the sample preparation cartridge module 208 may be divided into a first region 232 and a second region 234, divided by a barrier 236. A sample 238 is received into the sample preparation cartridge module 208 via the port 220, and may reside at a bottom of the first region 232, at the barrier 236. The sample preparation cartridge module 208 may further comprise an agitator 240 in the first region 232 which may be actuated via a mixer actuator 242 and an actuator 244, and the like of the device 200 as described below. In particular, the mixer actuator 242 may include a servomotor and/or servomotors, and the like, to move/rotate the actuator 244 to mix the sample 238 via the agitator 240, while the sample 238 is heated, as described below.

For example, the cassette 206 may be moved, along the vertical carriage guide 226, via the cassette carriage 224, into a heating position for heating by heaters 102 (e.g. two heaters 102-1, 102-2) attached to respective mechanical devices 106 (e.g. mechanical devices 106-1, 106-2). As depicted in FIG. 2B, it is understood that the heaters 102 and the mechanical devices 106 are in rest positions (e.g. similar to the rest position 110).

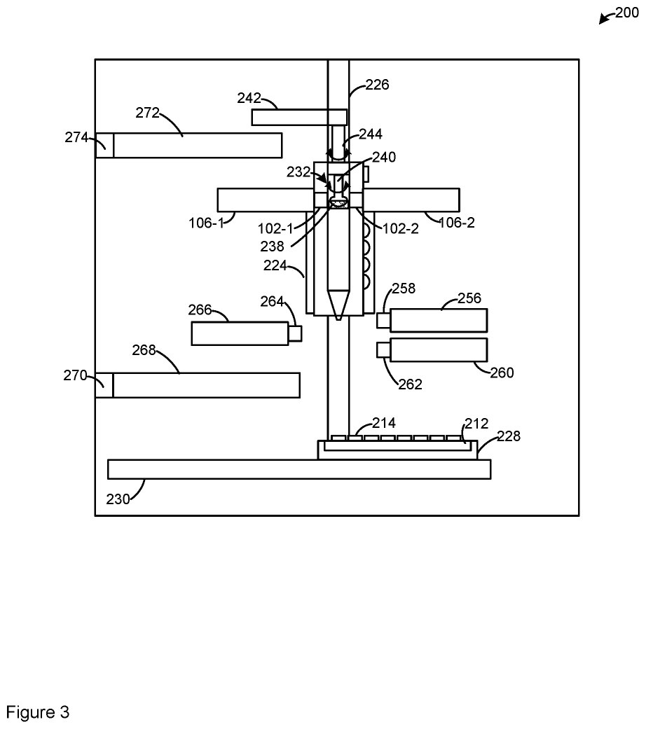

While not depicted in FIG. 2B, the device 200 is understood to include respective temperature sensors 104 at the heaters 102 and/or the mechanical devices 106 as in FIG. 1. Put another way, the device 100 may incorporate two devices 100, including two heaters 102, two sensors 104 and two mechanical devices 106, the heaters 102 and the mechanical devices 106 positioned to engage the sample preparation cartridge module 208 from opposite directions for example when the cassette 206 is loaded into the cassette carriage 224, and the cassette carriage 224 is in a heating position. In the heating position, shown in FIG. 3, described below, the sample 238 in the sample preparation cartridge module 208 is positioned along the vertical carriage guide 226 such that the sample preparation cartridge module 208 is at a sample position 108 of the heaters 102.

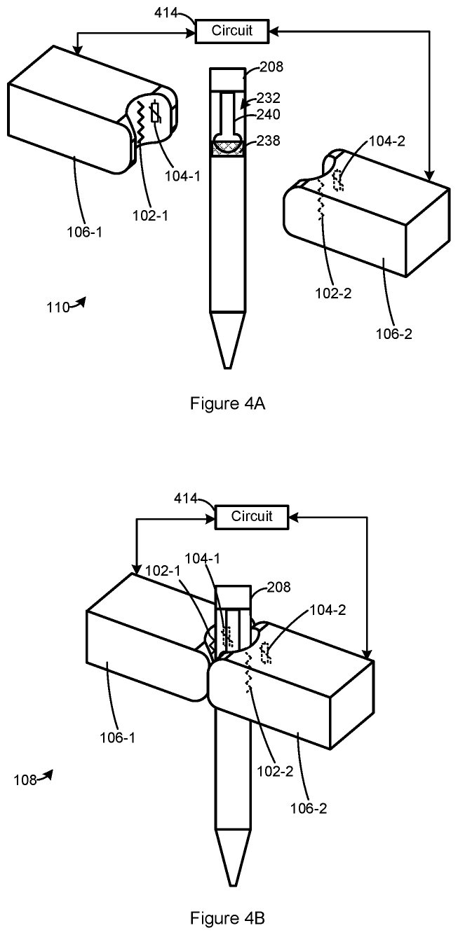

The heaters 102, mechanical devices 106 and temperature sensors 104 thereof are described in further detail with respect to FIG. 3, FIG. 4A and FIG. 4B. Furthermore, while not depicted, the device 200 may include a circuit or circuits similar to the circuit 114 but which controls the two heaters 102, the two mechanical devices 106 based on feedback from two temperature sensors 104.

When the cassette 206 is moved to a heating position of the device 200, the heaters 102 are about generally aligned with the sample 238 (e.g. perpendicularly), and the heaters 102 may be moved into respective sample positions to heat the sample 238, while the agitator 240 is actuated by the actuator 244 to agitate and/or mix the sample 238 while it is being heated, for example to promote lysis in cells of the sample 238. As such, the actuator 244 itself is understood to be further moved by the mixer actuator 242 into a position to agitate and/or mix the sample 238, while it is being heated, and actuated by the mixer actuator 242 which may comprise any suitable combination of motors for moving and turning the actuator 244. Alternatively, as will be explained in further detail below, the first region 232 may include magnetizing microparticles (including, but not limited to, paramagnetic microparticles) having surfaces to which biological components of interest, expelled by cells of the sample 238 due to lysis, bond; hence, in some examples, the actuator 244 may comprise a magnetic agitating device which agitates the sample 238 during lysis by applying a changing magnetic field to the first region 232 to move the magnetizing microparticles. In such examples, the agitator 240 may be omitted from the sample preparation cartridge module 208.

However, as depicted, it is understood that the agitator 240 is generally configured to mate with the actuator 244; for example, as depicted, the agitator 240 may be attached to a pressure source 250, such as a plunger, and the like, an outer surface of which may be used to both mate with the actuator 244, to actuate the agitator 240, and move the sample 238 to the second region 234, for example by applying pressure to the pressure source 250 via the actuator 244 to break the barrier 236.

Once lysis is performed on the sample 238, biological components of interest may be released from the sample 238 and bond to the magnetizing microparticles. While not depicted, the second region 234 may further include a wash buffer which may be mixed with the biological components of interest bonded to the magnetizing microparticles (e.g. when plunged into the second region 234), by actuation of a suitable reservoir 252 of a plurality of reservoirs 252 that perform different functions for the sample preparation cartridge module 208; such reservoirs 252 may be alternatively referred to as blisters and/or pouches, and the like. For example, one reservoir 252 may hold the wash buffer, another reservoir 252 may hold chemicals to stabilize biological components of interest, another reservoir 252 may hold a grease barrier, and yet another reservoir 252 may be for dispensing the processed sample 238, including the biological components of interest bonded to the magnetizing microparticles, into a well 214, for example via a needle and/or tip 254 of the sample preparation cartridge module 208.

As mentioned above, the cassette 206 may hold a plurality of sample preparation cartridge modules 208 and hence the device 200 may include various devices for actuating a plurality of corresponding reservoirs 252 (e.g. concurrently) on a plurality of sample preparation cartridge modules 208 and devices for actuating individual reservoirs 252 (e.g. independent of each other) on the sample preparation cartridge modules 208. For example, as depicted, the device 200 may include a multiple reservoir actuator 256 including a plurality of reservoir tips 258 (though only one is depicted) which may be used to actuate a plurality of corresponding reservoirs 252 (e.g. concurrently) on a plurality of sample preparation cartridge modules 208, for example to concurrently introduce the wash buffer, or the stabilizing chemicals or the grease barrier into the second regions 234 of the plurality of sample preparation cartridge modules 208. However, the device 200 may include a plurality of single reservoir actuators 260 (though only one is depicted) including respective reservoir tips 262 (though, again, only one is depicted), for independently actuating respective reservoirs 252 at the plurality of sample preparation cartridge modules 208 to independently dispense samples 238 into respective wells 214 via respective tips 262. In other examples, the device 200 may comprise one single reservoir actuator 260 including one reservoir tip 262 that is movable within the device 200 between sample preparation cartridge modules 208.

It is understood that the cassette carriage 224 may be moved into various suitable positions along the vertical carriage guide 226 relative to other components of the device 200, to effect actuation of the pressure source 250 and/or actuation of respective reservoirs 252 by the reservoir actuators 256, 260.

The device 200 and/or the sample preparation cartridge module 208 may include other suitable components. For example, the sample preparation cartridge module 208 may include the magnetizing microparticles in the first region 232 which have surfaces treated to bond to biological components of interest from the sample 238 when heated. Furthermore, the second region 234 may include a wash buffer density gradient, when the wash buffer is introduced into the second region 234; in particular, the second region 234 may comprise a fluid density gradient which isolates and/or purifies the biological components of interest bonded to the magnetizing microparticles. As depicted, the device 200 may include a magnet 264, which may be actuated via a magnetic actuator 266 to move the magnet 264 adjacent the sample preparation cartridge module 208 as the sample preparation cartridge module 208 is moved along the vertical carriage guide 226, for example to attract the magnetizing microparticles in the sample 238 to move the sample 238, biological components of interest bonded to the magnetizing microparticles, through the wash buffer density gradient in the second region 234 to isolate and/or purify the biological components of interest bonded to the magnetizing microparticles and/or move the sample 238, with biological components of interest bonded to the magnetizing microparticles, to the tip 254.

As depicted, the device 200 further includes a cooler and/or air-intake port 268 and/or tube which may include a fan, and the like (not depicted) for drawing air into the device 200 via a filter 270, and an exhaust port 272 (which may also include a fan) for expelling air drawn into the device 200 via the cooler port 268 via a respective filter 274. In particular, the ports 268, 272 may provide passive and/or active cooling at the device 200 to cool the sample 238 when heated. Furthermore, the ports 268, 272 may be located in any respective suitable positions at the device 200. Furthermore, when such cooling is active, a fan or fans of the ports 268, 272 may be controlled when cooling sample preparation cartridge modules as described herein.

Finally, once the samples 238 are processed as described, the cassette carriage 224 may be moved into a dispensing position relative to the wells 214 to dispense samples 238 into the wells 214. Thereafter, the wells 214 containing the processed samples 238, including the biological components of interest, may be moved out of the device 200 (e.g. via the access door 210), and transferred to a PCR assay device, and the like.

For clarity, attention is briefly directed to FIG. 3 which is substantially similar to FIG. 2B with like components having like numbers. However, in FIG. 3, the cassette 206 has been loaded into the cassette carriage 224, and lowered to a heating position relative to the heaters 102, and the cassette access door 204 has been closed. Furthermore, the heaters 102 have been moved, via the mechanical devices 106, to engage at least a portion of the first region 232 of the sample preparation cartridge module 208 to heat the sample 238. As such, in FIG. 3, it is understood that the heaters 102 and mechanical devices 106 are in sample positions (e.g. similar to the sample position 108).

Furthermore, the mixer actuator 242 has been moved to cause the actuator 244 to engage the agitator 240 and the mixer actuator 242 is being controlled (e.g. via processor and/or a circuit of the device 200) to turn the actuator 244, which turns the agitator 240 to mix the sample 238.

For completeness, FIG. 3 also depicts the well holder 212 and wells 214 loaded into the well carriage 228, with the horizontal carriage guide 230 moved into the device 200, with the well access door 210 closed.

Attention is next directed to FIG. 4A and FIG. 4B which respectively show the heaters 102-1, 102-2 and the mechanical devices 106-1, 106-2 of the device 200 in rest positions 110 (e.g. as in FIG. 2A) and sample positions 108 (e.g. as in FIG. 3). FIG. 4A and FIG. 4B further depict a sample preparation cartridge module 208 at the sample positions 108 such that, in FIG. 4B, the heaters 102 engage the sample preparation cartridge module 208 from opposite sides, and in particular the heaters 102 engage a portion of the first region 232 of the sample preparation cartridge module 208 to heat the sample 238. Hence, it is understood in FIG. 4A and FIG. 4B that the cassette carriage 224 (not depicted) has been moved to a heating position in the device 200 such that the first region 232 of the sample preparation cartridge module 208, containing the sample 238, has been moved between the heaters 102 while in the rest positions 110 and then the mechanical devices 106 have been controlled, for example by a circuit 414, to move the heaters 102 to the sample positions 108 to engage the sample preparation cartridge module 208 and heat the sample 238. The circuit 414 is understood to be similar to the circuit 414 but adapted to control positions of two heaters 102 by controlling respective mechanical devices 106, based on feedback from respective temperature sensors 104 (e.g. sensors 104-1, 104-2) located with the heaters 102.

In FIG. 4A and FIG. 4B, the heater 102-2 and the sensor 104-2 are depicted in dashed lines to show a position thereof at the mechanical device 106-2, as the heater 102-2 and the sensor 104-2 are understood to be seen “through” the mechanical device 106-2 due to the angle of perspective. Similarly, in FIG. 4B, the sensor 104-1 is depicted in dashed lines to show a position of the sensor 104-1 relative to the sample preparation cartridge module 208, as the sensor 104-1 is understood to be seen “through” the sample preparation cartridge module 208.

While not depicted, in FIG. 4B, the agitator 240 may be controlled to mix the sample 238 during heating (e.g. as shown in FIG. 3). Indeed, it is understood that in FIG. 4A the heaters 102 may be off, to either not heat the sample 238 and/or to allow the sample 238 to cool after heating. Such cooling may occur via airflow via the ports 268, 272, and fans thereof, when present, may be controlled by the device 200 to increase or decrease speed, to assist with such cooling. In contrast, in FIG. 4B, the heaters 102 may be on to heat the sample 238.

As such, FIG. 4A may represent the position of the heaters 102 before and after heating, while FIG. 4B may represent the position of the heaters 102 during heating. Furthermore, while in FIG. 4B both heaters 102 are controlled to engage the sample preparation cartridge module 208, in other examples, one heater 102 may be controlled to engage the sample preparation cartridge module 208 or both heaters 102 may be controlled to engage the sample preparation cartridge module 208.

Control of the heaters 102 and the mechanical devices 106 is further understood to occur via the circuit 414 which may include a processor and have access to a memory storing various parameters as will be next described.

FIG. 5 is a block diagram of control components of the device 200 that includes the circuit 414, and which may include a processor 500 which is incorporated into the circuit 414 or, as depicted, separate from the circuit 414. While other components of the device 200 are not depicted, they are nonetheless understood to be present.

The device 200 further depicts a computer-readable medium and/or memory 502. The memory 502 includes instructions that, when implemented by the processor 500 and/or the circuit 414, cause the processor 500 and/or the circuit 414 to implement mechanical heating control at the device 200. The memory 502 may be a computer-readable medium, such as a volatile computer-readable medium (e.g., volatile RAM, a processor cache, a processor register, etc.), a non-volatile computer-readable medium (e.g., a magnetic storage device, an optical storage device, a paper storage device, flash memory, read-only memory, non-volatile RAM, etc.), and/or the like. The processor 500 may be a general-purpose processor or special purpose logic, such as a microprocessor (e.g., a central processing unit, a graphics processing unit, etc.), a digital signal processor, a microcontroller, an ASIC, an FPGA, a PAL, a PLA, a PLD, etc. The memory 502 or the processor 500 may be distributed among a plurality of computer-readable media or a plurality of processors with the circuit 414 integrated accordingly.

The memory 502 includes instructions 504 (e.g. heating instructions) which may be provided in the form of a module and/or software modules (e.g., as used herein, a “module” and/or “software module” is a set of instructions that when executed or interpreted by a processor or stored at a processor-readable medium realizes a component or performs a method). Hence, for example, the instructions 504 may alternatively be replaced with an instruction module.

In particular, the instructions 504, when implemented by the processor 500 and/or the circuit 414, cause the processor 500 and/or the circuit 414 to control the mechanical devices 106 to move the heaters 102 based on feedback from the sensors 104, as well to control the heaters 102 to be on or off, and/or a power level thereof.

For example, as depicted, the memory 502 further stores given temperatures 506 to which the heaters 102 are to be controlled to heat the sample preparation cartridge module 208; similarly, as depicted, the memory 502 further stores a heating cycle 508 which may comprise an order of the given temperatures 506, as well time periods to which the sample preparation cartridge module 208 is to be heated to the given temperatures 506, and cooling time periods, for example lengths of time that the sample preparation cartridge module 208 is be cooled between being heated to the given temperatures 506. The given temperatures 506 and/or the heating cycle 508 may be programmed at the device 200 via the input device 218, and/or at a factory.

As depicted, the memory 502 further stores temperature conditions 510 for controlling the position of the heaters 102 based on feedback from the temperature sensors 104, as described herein. For example, the temperature conditions 510 may comprise a threshold temperature at which the heaters 102 are to be moved to respective rest positions 110 and, optionally, a given time period for which the threshold temperature is measured by the sensors 104.

While not depicted, the memory 502 may further store instructions for implementing other functionality of the device 200 including, but not limited to, movement of the carriage 224, the horizontal carriage guide 230, the well carriage 228, the mixer actuator 242, the actuator 244, the reservoir actuators 256, 260, the magnetic actuator 266 etc.

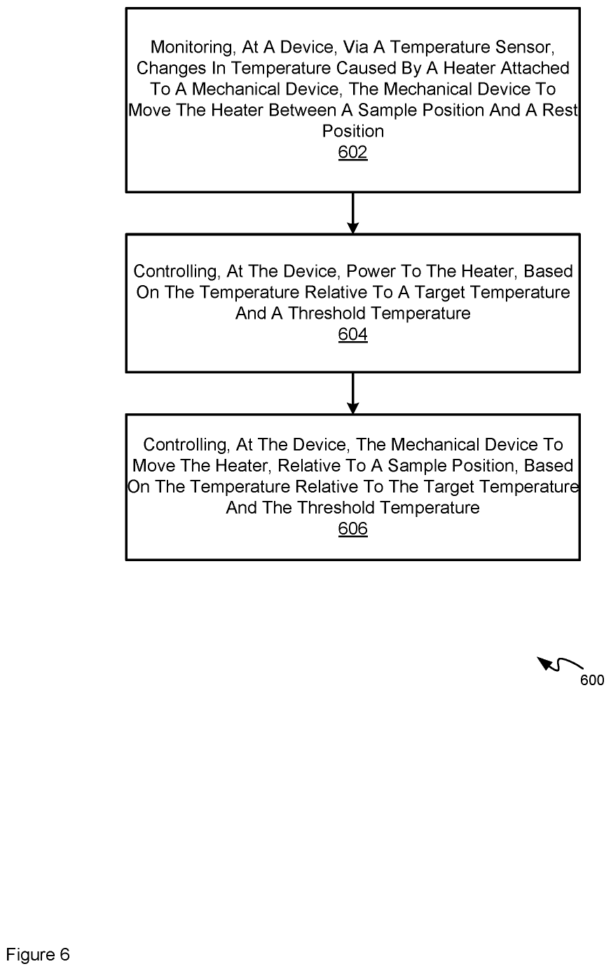

Referring to FIG. 6, a flow diagram of an example method 600 to perform mechanical heating control is depicted. In order to assist in the explanation of method 600, it will be assumed that method 600 may be performed with the device 200 (e.g. via the circuit 414 and/or processor 500 implementing the heating instructions 504). The method 600 may be one way in which the device 200 may be configured. Furthermore, the following discussion of method 600 may lead to a further understanding of the device 200, and its various components. Furthermore, it is to be emphasized, that method 600 may not be performed in the exact sequence as shown, and various blocks may be performed in parallel rather than in sequence, or in a different sequence altogether. Furthermore, it is to be emphasized that the method 600 may alternatively be performed with the device 100 and/or the circuit 114 thereof.

Furthermore, while the method 600 is described with respect to one respect to one heater 102, one temperature sensor 104, and one mechanical device 106, the method 600 may be implemented for the pair of heaters 102, temperature sensors 104, and mechanical devices 106 of the device 200.

Beginning at a block 602, the device 200 monitors, via a temperature sensor 104, changes in temperature caused by a heater 102 attached to a mechanical device 106, the mechanical device 106 to move the heater between a sample position 108 and a rest position 110.

At a block 604, the device 200 controls power to the heater 102, based on the temperature relative to a target temperature and a threshold temperature. The target temperature may be a given temperature 506 and a threshold temperature may be a threshold temperature of the temperature conditions 510.

At a block 606, the device 200 controls the mechanical device 106 to move the heater 102, relative to a sample position 108, based on the temperature relative to the target temperature and the threshold temperature.

Put another way, at the block 604 and the block 606, the device 200 generally controls power to the heater 102 and a position of the heater 102 to heat a sample preparation cartridge module 208 at a sample position 108 to the target temperature while also preventing the sample preparation cartridge module 208 from reaching or exceed the threshold temperature, as described above.

For example, at the block 604, the device 200 may control the power to the heater 102, based on the temperature relative to the target temperature and the threshold temperature, by: in response to the temperature reaching the threshold temperature, turning the heater 102 off until the temperature is at or below the target temperature, for example to prevent overheating of a sample preparation cartridge module 208.

Similarly, at the block 606, the device 200 may control the mechanical device 106 to move the heater 102, based on the temperature, relative to the target temperature and the threshold temperature, by: in response to the temperature reaching the threshold temperature, moving the heater 102 way from the sample position 108 (e.g. towards a rest position 110) until the temperature is at or below the target temperature.

Furthermore, as described herein, moving the heater 102 and/or turning the heater 102 off may be based on a time-based measurement of temperature. In particular, the method 600 may further comprise the device 200, in response to the temperature measured by the sensor 104 reaching or exceeding the threshold temperature for a given time period: moving the heater 102 away from the sample position 108 (e.g. at the block 606); and turning the heater 102 off (e.g. at the block 604).

In some examples, the method 600 may further comprise the device 200, in response to the temperature reaching or exceeding the threshold temperature for a given time period: controlling a notification device to provide a notification of a fault. For example, the device 200 may control a touch screen of the input device 218 to provide a visual notification of a fault (e.g. as the touch screen of the input device 218 may comprise a notification device). Similarly, while not depicted, the device 200 may comprise a notification device in the form a speaker which may be controlled to provide an aural notification of a fault. Similarly, while not depicted, the device 200 may comprise network connector and/or a transceiver to communicate with a remote notification device, and the like, for example a mobile device of a user of the device 200; in these examples, the device 200 may transmit a notification of a fault to the remote notification device.

The heaters 102, temperature sensors 104, and mechanical devices 106 provided herein may be used in other ways. For example, as there are may be two heaters 102 and two mechanical devices 106, the two heaters 102 may be heated to different temperatures and moved to respective sample positions 108 to rapidly transition sample preparation cartridge modules 208 between two (or more than two) temperatures.

Similarly, a first heater 102 of a first mechanical device 106 may be one while a second heater 102 of a second mechanical device 106 may be off; in these examples, the first heater 102 of the first mechanical device 106 may be used to heat a sample preparation cartridge module 208, and then the first mechanical device 106 may be removed from the sample preparation cartridge module 208, and the second mechanical device 106, with the off heater 102, may be moved to the sample preparation cartridge module 208 to act as a heatsink to cool the sample preparation cartridge module 208.

Furthermore, power used by the heaters 102, and temperature sensed by the temperature sensors 104, may be sensed/determined over time which may be used to estimate thermal dynamics of sample preparation cartridge modules 208. For example, heat resistance and/or heat capacitance of a mechanical device 106, which may be measured via power used by the heaters 102, and temperature sensed by the temperature sensors 104 may be an indicator of presence of a sample preparation cartridge module 208 at a sample position 108 and/or thermal properties/dynamics thereof. In one example, high thermal resistance at a mechanical device 106 may indicate air bubbles in a sample preparation cartridge module 208 at a sample position 108. Hence, problem sample preparation cartridge modules 208 may be detected in this manner (e.g. and an alert thereof may be provided at the device 200 (e.g. at the touch screen display of the input device 218, and the like).

It should be recognized that features and aspects of the various examples provided above may be combined into further examples that also fall within the scope of the present disclosure.

1. A device comprising:
a heater;
a temperature sensor to monitor changes in temperature caused by the heater;
a mechanical device to move the heater between a sample position and a rest position; and
a circuit communicatively coupled to the heater, the temperature sensor, and the mechanical device, the circuit configured to:
control the mechanical device to move the heater to the sample position;
control the heater to heat at the sample position;
monitor, via the temperature sensor, the temperature; and
in response to the temperature meeting a temperature condition, control the mechanical device to move the heater from the sample position to the rest position.

2. The device of claim 1, wherein the circuit is further configured to, in response to the temperature meeting the temperature condition, control the heater to stop heating.

3. The device of claim 1, wherein the temperature sensor is located at the heater.

4. The device of claim 1, wherein the mechanical device comprises (i) a robotic arm or (ii) an arm and a servomotor, and wherein the heater is located at an end effector position of the mechanical device.

5. The device of claim 1, wherein the heater is further configured to heat a sample preparation cartridge module at the sample position to effect a biological or chemical process in the sample preparation cartridge module.

6. A device comprising:
a heater;
a temperature sensor to monitor changes in temperature caused by the heater;
a mechanical device to move the heater between a sample position and a rest position; and
a circuit communicatively coupled to the heater, the temperature sensor, and the mechanical device, the circuit configured to:
control the heater to generate heat;
monitor, via the temperature sensor, the temperature; and
control the mechanical device to adjust a position of the heater, relative to the sample position and the rest position, based on the temperature, to control a heating cycle.

7. The device of claim 6, wherein the circuit is further configured to control the mechanical device to adjust the position of the heater, to control the heating cycle, by adjusting a distance of the heater from the sample position.

8. The device of claim 6, wherein the circuit is further configured to control the mechanical device to adjust the position of the heater, to control the heating cycle, by adjusting a mechanical pressure or a mechanical force at the sample position.

9. The device of claim 6, wherein the circuit is further configured to control the mechanical device to adjust the position of the heater, to control the heating cycle, by controlling a time period that the heater is at the sample position.

10. The device of claim 6, wherein the circuit is further configured to control the heating cycle by controlling power to the heater.

11. A method comprising:
monitoring, at a device, via a temperature sensor, changes in temperature caused by a heater attached to a mechanical device, the mechanical device configured to move the heater between a sample position and a rest position;
controlling, at the device, power to the heater, based on the temperature relative to a target temperature and a threshold temperature; and
controlling, at the device, the mechanical device to move the heater, relative to the sample position, based on the temperature relative to the target temperature and the threshold temperature.

12. The method of claim 11, wherein controlling the mechanical device to move the heater, based on the temperature, relative to the target temperature and the threshold temperature, comprises:
in response to the temperature reaching the threshold temperature, moving the heater away from the sample position until the temperature is at or below the target temperature.

13. The method of claim 11, wherein controlling the power to the heater, based on the temperature relative to the target temperature and the threshold temperature, comprises:
in response to the temperature reaching the threshold temperature, turning the heater off until the temperature is at or below the target temperature.

14. The method of claim 11, further comprising, in response to the temperature reaching or exceeding the threshold temperature for a given time period:
moving the heater away from the sample position; and
turning the heater off.

15. The method of claim 11, further comprising, in response to the temperature reaching or exceeding the threshold temperature for a given time period:
controlling a notification device to provide a notification of a fault.
